(19) United States
(12) Patent Application Publication
(10) Pub. No.: US 2024/0174967 A1
(43) Pub. Date: May 30, 2024

(54) Generic sterile sensor system on mobile mast

(71) Applicants: Commissariat à l'Energie Atomique et aux Energies Alternatives, PARIS (FR); GLOBAL PROCESS CONCEPT, PERIGNY (FR)

(72) Inventors: Tristan CAROFF, GRENOBLE (FR); Sébastien BRULAIS, GRENOBLE (FR); Martin GAUROY, GRENOBLE (FR); Jean-Yves BURLET, GRENOBLE (FR); Clément GARAFFA, GRENOBLE (FR); Zsolt POPSE, PERIGNY (FR)

(21) Appl. No.: 18/518,769

(22) Filed: Nov. 24, 2023

(30) Foreign Application Priority Data
Nov. 25, 2022 (FR) 2212356

(51) Int. Cl. C12M 1/34; C12M 3/00

(52) U.S. Cl. CPC C12M 41/00; C12M 23/46

(57) ABSTRACT

A device for analyzing at least one parameter of a chemical or biochemical or biological reaction in a reaction container, the analysis device includes an arm intended to be fastened by means of a sliding link against a face of a wall of the reaction container by means of a static end, the arm having a degree of freedom in translation in relation to the wall, a sensor designed to measure the at least one parameter, the sensor being fastened to a free end of the arm, a drive device designed to cause an axial displacement of the arm along the degree of freedom, a hermetic isolating device for isolating the arm from the reaction, the hermetic isolating device joining the arm to the face of the wall.

### CROSS-REFERENCE TO RELATED APPLICATION

This application claims priority to foreign French patent application No. FR 2212356, filed on Nov. 25, 2022, the disclosure of which is incorporated by reference in its entirety.

### FIELD OF THE INVENTION

The invention relates to the pharmaceutical, cosmetics, biomass production, and agri-food sectors.

### BACKGROUND

In particular, the invention relates to a device for analyzing and monitoring at least one parameter of a chemical or biochemical or biological reaction in a reaction container.

The multiplication or culture of micro-organisms is widely used in the pharmaceutical, cosmetology and agri-food industries to produce biomass, for the production of a metabolite or the bioconversion of a target molecule, as well as for wine making and brewing.

This multiplication is advantageously executed in a bioreactor that enables culture conditions to be controlled, and more specifically reaction parameters such as temperature, pH, oxygen level or biomass growth rate.

In a known manner, a bioreactor comprises a reaction container usually containing a stirrer and/or an aerator intended respectively to homogenize and oxygenate the reaction medium. The bioreactor further comprises a thermal jacket that ensures thermalization of the reaction medium. Such a bioreactor is also provided with sensors, for example temperature, pH and dissolved-oxygen sensors, to monitor the state of the culture and, where applicable, to adjust the culture conditions within the reaction container, preferably in real time.

These sensors are usually mounted statically on rods immersed in the reaction medium.

However, this sensor fastening has several drawbacks.

This is because the sensors held statically only sample the reaction medium locally, i.e. in the vicinity of the sensors themselves, and do not detect all potential non-homogeneity in the reaction medium occurring away from the sensors. The data collected by these sensors therefore do not enable optimal adjustment of the reaction parameters.

Consequently, a “mobile sensor” has been envisaged to better detect non-homogeneity in the reaction medium. Such a sensor can notably be seated in a capsule immersed in the reaction medium. The capsule is then subjected to the currents created by the stirrer, and thereby exposed to different zones of the reaction medium.

Nonetheless, these capsules move freely about the bioreactor, and therefore have the drawback of coming into regular contact with the walls of the bioreactor or with blades of the stirrer, and may be damaged as a result.

Furthermore, the path taken by the capsules in the reaction medium is random and does not enable coverage of the entire bioreactor.

Therefore, as disclosed in document EP3967743A1, it has been envisaged to control the movement of the mobile sensor using a mast fastened to two opposing walls of the reaction container. The analysis kit described in document EP3967743A1 thus proposes a hollow mast fastened against two opposing walls of the reaction container which comprises a mobile sensor that can move in translation along the mast by means of a magnetic link to the mast. The sensor is then moveable in translation along a transverse axis closed by the mast.

However, such a facility has other drawbacks. This is because there are two main types of bioreactors: stainless steel or glass bioreactors and flexible single-use bioreactors (SUB). The main difference is that the stainless steel or glass bioreactor can be used many times and therefore requires a complex clean (acid/base) to remove all organisms before the following production, while flexible single-use bioreactors are disposable after a single use and have the advantage of ensuring sterility without cleaning. Flexible single-use bioreactors today account for approximately 80% of production for the pharmaceutical industry. The load-bearing structure of these disposable bioreactors is less rigid than stainless steel or glass bioreactors.

Consequently, disposable bioreactors are not usually designed to receive a device such as the one disclosed in document EP3967743A1. More specifically, the walls of the reaction containers of the flexible single-use bioreactors are not suitable for the dual fastening of the mast, and therefore need to be adapted to be able to incorporate the mast. It is then complicated to adapt such an analysis device for flexible single-use bioreactors by adding a fastening at the top of the reaction container and a fastening at the bottom of the reaction container.

Furthermore, such devices have sterility issues when inserting the system into the bioreactor.

### SUMMARY OF THE INVENTION

The invention is intended to overcome some or all of the aforementioned problems by proposing a generic sterile sensor system integrated onto a mobile mast for measuring physiological or physical magnitudes over the entire height of a bioreactor, the mast being easy to integrate in any the reaction container. The analysis device according to the invention also has the advantage of enabling the sensor and the mobile mast to be inserted hermetically and without impacting the reaction medium.

For this purpose, the invention relates to a device for analyzing at least one parameter of a chemical or biochemical or biological reaction in a reaction container, the analysis device comprising:
an arm intended to be fastened by means of a sliding link against a face of a wall of the reaction container by means of a static end, the arm having a degree of freedom in translation in relation to the wall of the reaction container,
a sensor designed to measure the at least one parameter, the sensor being fastened to a free end of the arm,
a drive device designed to cause an axial displacement of the arm along the degree of freedom,
a hermetic isolating device for isolating the arm from the chemical or biochemical or biological reaction, the hermetic isolating device joining the arm to the face of the wall of the reaction container.

According to one aspect of the invention, the hermetic isolating device is made of one of the following materials: silicone, a fluoroelastomer, polyether ether ketone, ethylene propylene diene monomer, polytetrafluoroethylene.

According to one aspect of the invention, the hermetic isolating device comprises a bellows.

According to one aspect of the invention, the wall comprises an inner face facing the reaction, the hermetic isolating device being arranged to join the arm to the inner face of the wall.

According to one aspect of the invention, the wall comprises an outer face facing an environment outside the reaction, the hermetic isolating device being arranged to join the arm to the outer face of the wall.

According to one aspect of the invention, the analysis device comprises control means for the arm that are connected to the drive device and arranged to control the displacement of the arm along the degree of freedom.

According to one aspect of the invention, the analysis device comprises means for transmitting data that can be measured by the sensor.

According to one aspect of the invention, the analysis device comprises a device for recording the data measured by the sensor.

According to one aspect of the invention, the sensor is a sensor able to measure one of the following data: pH, dissolved oxygen, carbon dioxide, electrical conductivity, redox potential or temperature.

According to one aspect of the invention, the arm is a telescopic arm.

According to one aspect of the invention, the hermetic isolating device comprises a seal positioned between the bellows and the face of the wall of the reaction container, the seal comprising a first sealing surface in contact with the face of the wall of the reaction container and a second sealing surface in contact with the bellows.

The invention also relates to a reaction container for a chemical or biochemical or biological reaction comprising the device for analyzing at least one parameter of the chemical or biochemical or biological reaction in the reaction container.

### BRIEF DESCRIPTION OF THE DRAWINGS

The invention will be better understood and other advantages will become apparent on reading the detailed description of an embodiment given by way of example, which description is illustrated by the appended drawing, in which:

FIG. 1 is a schematic view of an analysis and monitoring device according to the present invention,

FIG. 2 is a schematic view of the analysis and monitoring device in a preferred architecture,

FIG. 3 is a schematic view of a variant of the analysis and monitoring device in FIG. 2,

FIG. 4 shows a schematic view of a reaction container comprising two analysis devices according to the invention;

FIG. 5A is a schematic view of the analysis and monitoring device according to a first embodiment in which a hermetic isolating device is extended,

FIG. 5B is a schematic view of the analysis and monitoring device in FIG. 5A in which the hermetic isolating device is compressed,

FIG. 6A is a schematic view of the analysis and monitoring device according to a second embodiment in which the hermetic isolating device is extended,

FIG. 6B is a schematic view of the analysis and monitoring device in FIG. 6A in which the hermetic isolating device is compressed,

FIG. 7 is a detailed view of the analysis and monitoring device in FIGS. 6A and 6B,

FIG. 8 is a detailed view of the fastening of the arm of the analysis and monitoring device according to the invention,

FIG. 9 is a magnified view of a face of the hermetic container against which the hermetic isolating device according to the invention rests.

For the sake of clarity, the same elements bear the same reference signs in the various figures.

### DETAILED DESCRIPTION

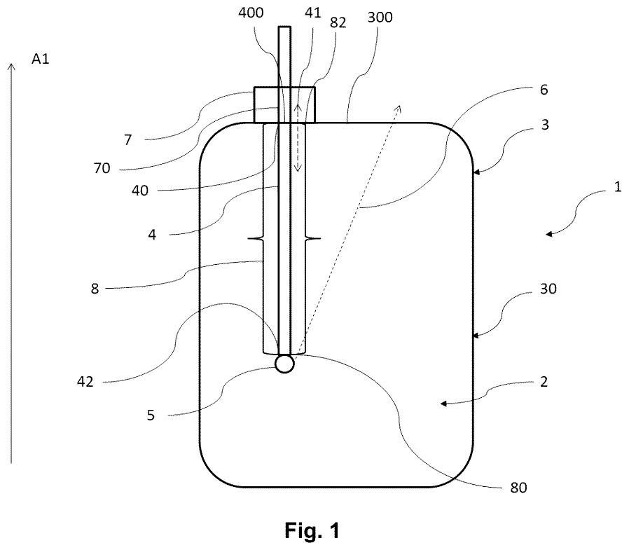

FIG. 1 is a schematic view of a device 1 for analyzing and monitoring at least one parameter of a chemical or biochemical or biological reaction 2 in a reaction container 3.

The analysis device 1 comprises an arm 4 intended to be fastened by means of a sliding link 41 against a face 300 of a wall 30 of the reaction container 3 by means of a static end 40. The arm 4 has a degree of freedom in translation in relation to the wall 30 of the reaction container 3. In other words, the arm 4 is designed to extend axially in relation to the wall 30 and locally in relation to the face 300 along a first axis A1. According to a preferred embodiment of the invention, the arm 4 moves in translation substantially perpendicularly to the face 300 of the wall 30.

In a variant, the arm 4 can move in translation along an axis secant with the wall 300. Any axis of translation that does not come into unwanted contact with a component such as the stirrer of the bioreactor may be used.

The arm 4 is therefore slidingly linked to the face 300 of the wall 30 of the reaction container 3, enabling it to be guided in translation along the first axis A1.

The analysis device 1 also comprises a sensor 5 designed to measure the at least one parameter of the reaction 2, the sensor 5 being fastened to a free end 42 of the arm 4 in contact with the reaction 2. This free end 42 is a moveable end because the arm 4 is moveable in translation. The free end 42 is therefore substantially opposite the face 300 of the wall 30. Furthermore, the sensor 5 is therefore intended to collect data from a reaction medium in which the sensor 5 can be immersed.

The analysis device 1 may also comprise several sensors 5 fastened to the free end 42 of the arm 4 to measure several parameters of the reaction 2.

Notably, the data collected by the sensor or sensors 5 may relate to the experiment conditions imposed on the reaction medium. The latter may be characteristics of acidity, stirring, and temperature. The collected data may also relate to the progress of the reaction 2 or phenomena occurring in the reaction medium.

By way of example, the sensor or sensors 5 may comprise at least one of the following elements: a pH measurement sensor, a temperature measurement sensor, a sensor for measuring a quantity of dissolved oxygen, a sensor for measuring a quantity of carbon dioxide or dissolved oxygen, a sensor able to measure electrical conductivity or redox potential, i.e. the reduction-oxidation reaction occurring inside the reaction 2. The invention is not limited to only these sensors, and the person skilled in the art will be able to implement any other sensors depending on the reaction parameters to be monitored.

The analysis device 1 also comprises means 6 for transmitting data that can be measured by the sensor 5. The data transmission means 6 therefore enable the data collected by the sensor 5 to be sent to a platform, enabling real-time analysis of the evolution of the reaction medium.

Furthermore, the analysis device 1 comprises a drive device 7 designed to cause the axial displacement of the arm 4 along the degree of freedom. In other words, the drive device 7 can cause the translational movement of the arm 4 and therefore the sensor 5 along the first axis A1 inside the reaction container 3. Furthermore, according to a preferred embodiment, the drive device 7 may comprise a guide channel 70 for the arm 4 that is traversed by the arm 4 moving in translation along the first axis A1, enabling the movement of the arm 4 to be channeled.

The drive device 7 thus enables the sensor 5 to be used to measure the parameter of the chemical or biochemical or biological reaction 2 in the reaction container 3. This measurement can advantageously be taken at different levels or different heights in the reaction container 3 on account of the drive device 7, which enables controlled translational movement of the arm 4 and of the sensor 5. The drive device may preferably be a belt-driven linear actuator. This type of actuator has the advantage of enabling control of the translational movements of the arm 4 and of the sensor 5 to enable the sensor 5 to measure a parameter in the reaction 2 at different positions, different levels or different heights in the reaction container 3.

In a variant, the arm 4 may also be guided in its translational movements by a double universal joint or a Silentbloc flexible coupling to ensure that the translational movement is not overstressed. This assembly keeps the arm 4 correctly aligned, withstands the radial load in the link between the arm 4 and the drive device 7 caused by the pressure of the reaction 2 on the arm 4, and ensures a seal between the inside of the hermetic isolating device 8 and the reaction 2 to prevent any contamination.

By way of example, the drive device 7 may be a ball screw motor, a pantograph, or a linear actuator.

In a variant, any motorized device causing a translational movement may be used.

Furthermore, the analysis device 1 comprises a hermetic isolating device 8 isolating the arm 4 from the chemical or biochemical or biological reaction 2. The hermetic isolating device 8 is arranged to join the arm 4 to the face 300 of the wall 30 of the reaction container 3. The hermetic isolating device 8 therefore enables the arm 4 to be isolated from the reaction 2 in the reaction container 3. This guarantees the sterility of the reaction 2, even while the arm 4 is in movement in the reaction container 3.

According to the invention, the hermetic isolating device 8 is arranged such as to prevent the ingress of any foreign bodies into the reaction 2 through an opening 400 formed in the static end 40 during translational movement of the arm 4. This is because, when the arm 4 moves in translation toward a face opposite the face 300 of the wall 30, resulting in an insertion movement of the arm 4 and the sensor 5 into the reaction container 3, any bodies foreign to the reaction 2 could become attached to the arm 4 and pass through the opening 400 in the wall 30 of the reaction container 3 and come into contact with the reaction medium and contaminate the reaction 2 underway. The hermetic isolating device 8 thus provides a sealing zone between the arm 4 and the reaction 2 under all translational movement positions along the first axis A1.

According to a preferred embodiment, the hermetic isolating device 8 comprises a first sealed end 80 joined to the free end 42 and a second sealed end 82 joined to the face 300 of the wall 30 in the vicinity of the static end 40 for example.

According to the invention, the hermetic isolating device 8 is made of one of the following materials: silicone, a fluoroelastomer, polyether ether ketone (PEEK), ethylene propylene diene monomer (EPDM), polytetrafluoroethylene (PTFE) or any other material biocompatible with the reaction 2 so as not to impact the reaction medium.

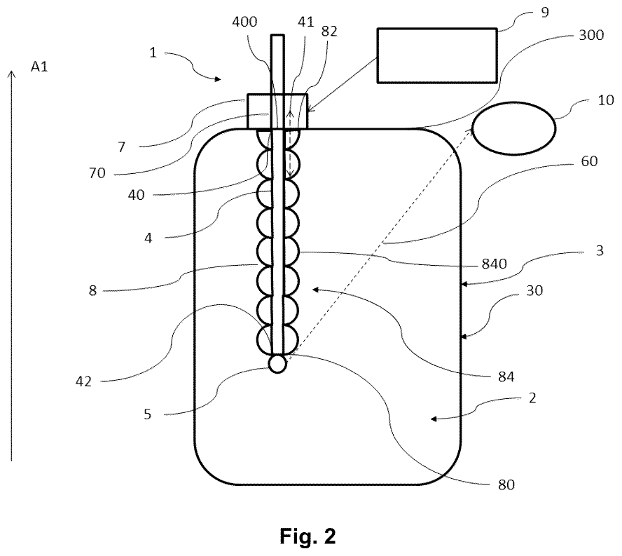

According to a preferred embodiment, the hermetic isolating device 8 comprises a bellows 84, as shown in FIG. 2. The bellows 84 has the advantage of being suited to each position of the arm 4. This is because a bellows 84 has an extendible elastic structure in the form of blisters 840 that can be stretched by extending each blister 840 and compressed by compressing each blister 840. The use of a bellows 84 adapted to the movement of the arm 4 therefore has the advantage of not impacting the reaction 2 during displacements of the arm 4 and the sensor 5, for example by generating unwanted local disturbances and current movements in the reaction 2. Furthermore, such a bellows 84 has the advantage of withstanding the many translational movements of the arm, and therefore the traction and compression forces to which it is subjected.

By way of example, the bellows 84 may have a cylindrical, rectangular or conical section.

In a variant, the hermetic isolating device 8 comprises an elastic cylindrical pocket that can withstand the traction and compression forces exerted by the translational movement of the arm 4.

Advantageously, the analysis device 1 may also comprise control means 9 for the arm 4 arranged to control the displacement of the arm 4 along the degree of freedom. More specifically, the control means 9 are connected to the drive device 7 such that the drive device 7 causes a translational movement of the arm 4 and of the sensor 5 along the first axis A1 in response to a command from the control means 9. The control means 9 thus enable the arm 4 to be controlled. By way of example, the typical linear translational speed of the arm 4 is in the order of 0.01 m/s to 1.5 m/s and the spatial resolution is in the order of 0.1 mm to 1 cm, depending on the type of linear actuator or motor used as the drive device 7.

The control means 9 can then be used to pre-set the successive translational movement or movements of the arm 4 in the reaction container 3, or be controlled in real time by a user.

The analysis device 1 may also comprise a device 10 for recording the data measured by the sensor 5. The recording device 10 then communicates with the sensor 5 using the data transmission means 6. Furthermore, the data transmission means 6 may be wireless transmission means 60, for example Wi-Fi, Zigbee or Bluetooth.

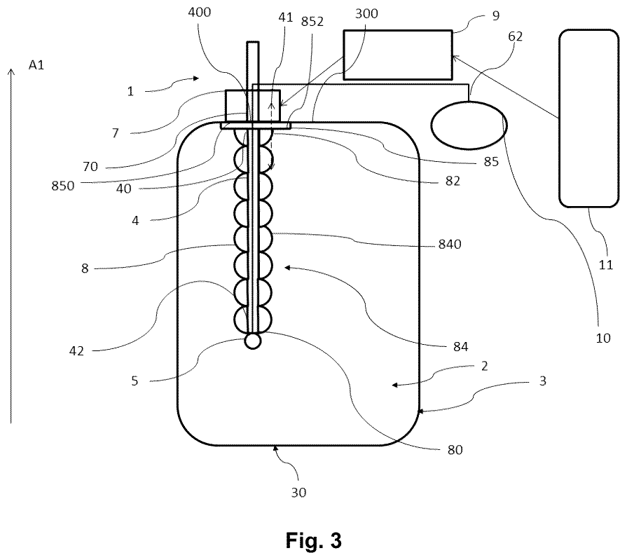

In a variant shown in FIG. 3, the data transmission means 6 may be wired transmission means 62 connecting the sensor 5 to the recording device 10, for example RS432, RS485, or RJ45.

The analysis device 1 may also comprise a power source 11 designed to power the control means 9 and the drive device 7.

Advantageously, the hermetic isolating device 8 may also comprise a seal 85 positioned between the bellows 84 or the cylindrical pocket and the face 300 of the wall 30 of the reaction container 3. Indeed, the seal 85 comprises a first sealing surface 850 in contact with the face 300 of the wall 30 of the reaction container 3 and a second sealing surface 852 in contact with the bellows 84 or with the cylindrical pocket. The seal 85 thus provides additional isolation at the opening 400.

Indeed, the fact of moving the arm 4 in translation, and more specifically inserting and withdrawing the arm 4 into and from the reaction medium 2 in the reaction container entails a significant risk of contaminating the reaction 2. The seal 85 thus prevents the bellows 84 or the cylindrical pocket from coming into contact with the reaction 2, thereby reducing the risk of contamination. In other words, the seal 85 creates an intermediate space or volume 800 between the reaction container 3 containing the reaction 2 and the environment outside the reaction container 3. As shown in FIG. 1 and FIG. 2, this intermediate volume 800 is the internal volume of the bellows 84 or the cylindrical pocket. This is the volume between the bellows 84 or the cylindrical pocket and the arm 4. This intermediate volume 800 inside the bellows 84 or the cylindrical pocket is never in contact with the reaction 2. This intermediate volume 800 inside the bellows 84 thus has the advantage of guarding the reaction 2 against any contamination outside the reaction container 3. All contaminants outside the reaction container 3 are then stopped by the seal 85 in the intermediate volume 800 in the bellows 84 or the cylindrical pocket.

The use of the seal 85 therefore enables the sensor 5 to take measurements at different levels in the reaction container 3 and in different positions by actuating the drive device 7. The drive device and the seal 85 thus enable the arm 4 and the sensor 5 to be moved in translation with no risk of contagion for the reaction 2.

In a variant, the seal 85 may be a wiper seal comprising a flexible inner lip bearing against the arm 4 and an outer lip fastened to the wall 30 of the reaction container 3. More specifically, the inner lip is pressed against the arm 4 so that the wiper seal creates a seal when the arm 4 is sliding. Furthermore, the wiper seal may also comprise several superposed internal lips pressed against the arm 4 to improve the seal of the wiper seal.

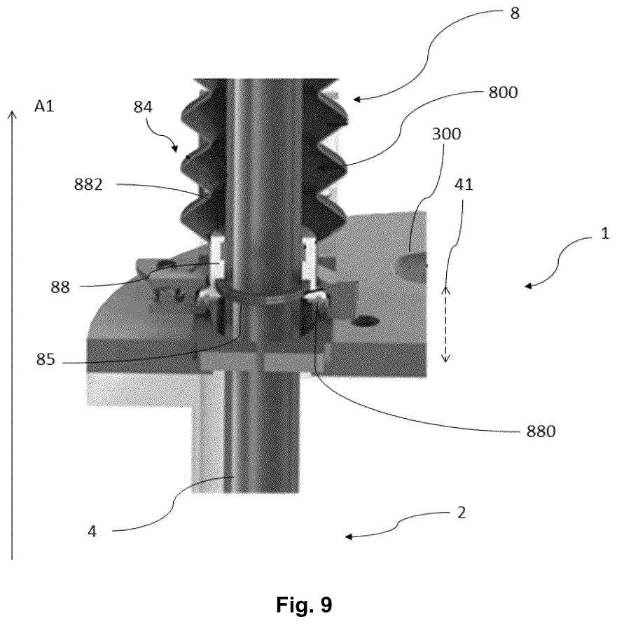

The hermetic isolating device 8 may also comprise a guide element 88, shown in FIG. 9. The guide element 88 bears against the face 300 along a first edge 880 of the guide element 88. The guide element 88 comprises a section 882 shaped to fit the arm 4. In the example illustrated, the arm 4 is cylindrical. The guide element then comprises a cylindrical open section 882 dimensioned to fit the arm 4. The arm 4 passes through the guide element 88 and the face 300 when moving in translation guided by the drive device 7. The arm 4 passes through the guide element 88 along the sliding link 41 in relation to the first axis A1. The guide element 88 is designed to guide the translational movement of the arm 4 parallel to the first axis A1 such that the translational movement of the arm 4 caused by the drive device 7 is exactly parallel to the first axis A1. The guide element 88 ensures that there is no relative movement of the arm 4 in relation to the face 300 in the plane parallel to the face 300 of the wall 30. The only movement between the arm 4 and the face 300 is then the translational movement along the first axis A1 caused by the sliding link 41 between the face 300 and the arm 4. The guide element 88 is then in contact with the arm 4 at the open section 882 thereof traversed by the arm 4, and guides the translational movement of the arm 4.

The seal 85 blocks external contaminants so that no contaminants can enter the reaction container 3 and the guide assembly 88 keeps the arm 4 precisely centered on the first axis A1, which improves the performance of the sealing system. Indeed, the absence of any relative movement between the arm 4 and the face 300 in the plane parallel to the face 300 ensures that there is no deformation of the seal 85 in the plane parallel to the face 300, i.e. radial deformations. Such radial deformations can limit the sealing capacity of the seal 85. Preventing relative movement between the arm 4 and the face 300 then helps to improve the seal of the hermetic isolating device 8.

The guide element 88 can be made of polyether ether ketone (PEEK), which is a rigid thermoplastic resistant to gamma radiation.

The seal 85 can be made of ethylene propylene diene monomer (EPDM), which has good elasticity.

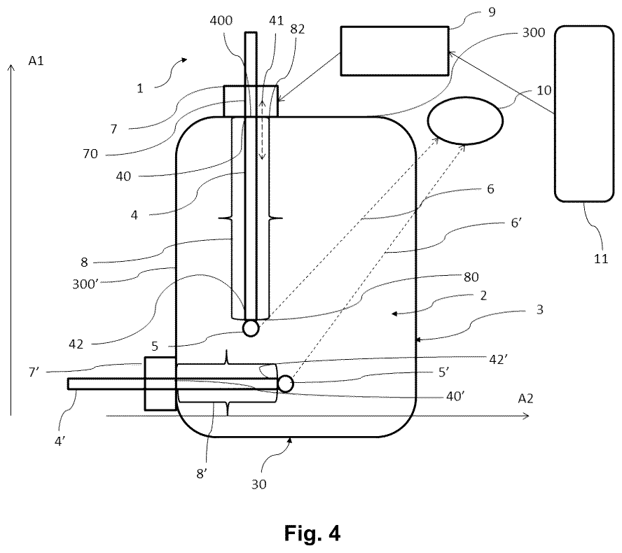

Several analysis devices 1 may also be used, as shown in FIG. 4. Indeed, the use of an arm 4 capable of moving in translation along the first axis A1 limits the acquisition of data by the sensor 5 to a height range, in the example illustrated, without observing the reaction medium along a second axis A2 that is at least secant and preferably perpendicular to the first axis A1 in a plane formed by the first axis A1 and the second axis A2.

The analysis device 1 can therefore comprise:
a second arm 4′ intended to be fastened to a second face 300′ of the wall 30 of the reaction container 3 by means of a static end 40′. The second arm 4′ also has a degree of freedom in translation in relation to the wall 30 of the reaction container 3. The second arm 4′ therefore moves in translation along the second axis A2.
a second sensor 5′ designed to measure at least one parameter, and fastened to a free end 42′ of the second arm 4′,
second means 6′ for transmitting data that can be measured by the sensor 5′ of the second arm 4′,
a second drive device 7′ designed to cause an axial displacement of the second arm 4′ along the degree of freedom of the second arm 4′ and therefore along the second axis A2,
and a second hermetic isolating device 8′ for isolating the second arm 4′ from the chemical or biochemical or biological reaction 2, designed to join the second arm 4′ to the second face 300′ of the wall 30 of the reaction container 3.

The first face 300 and the second face 300′ are two different faces of the wall 30 of the reaction container 3, and are preferably two faces that are perpendicular or at least secant.

In a variant, the second axis A2 can also be secant to the first axis A1 in the plane formed by the first axis A1 and by the second axis A2.

So as not to limit the movements of the arm 4 and of the second arm 4′, it is desirable for the arm 4 and the second arm 4′ to be remote from one another along an axis perpendicular to the first axis A1 and to the second axis A2.

In a variant, more than two arms having a degree of freedom in translation may be used in the analysis device 1.

In the remainder of the description, a single arm 4 is mentioned. However, the second arm 4′ or more than two arms may also be used.

The arm 4 may advantageously be a telescopic arm. A telescopic arm has the advantage of being compact.

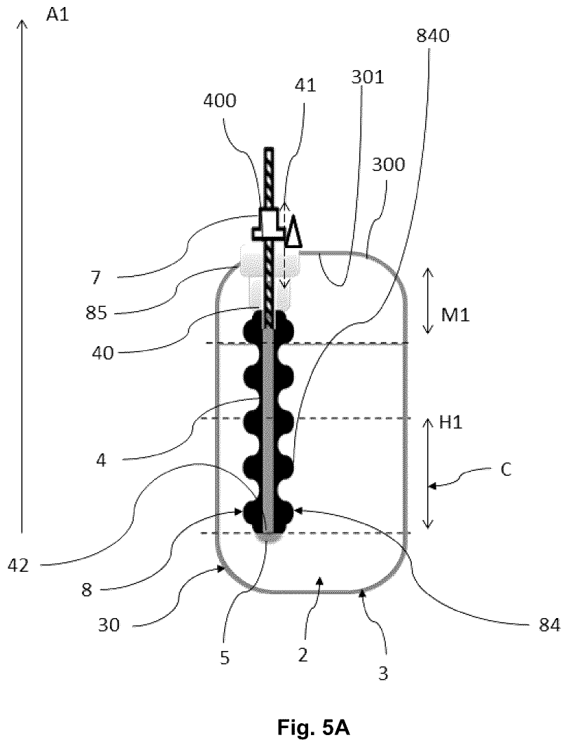

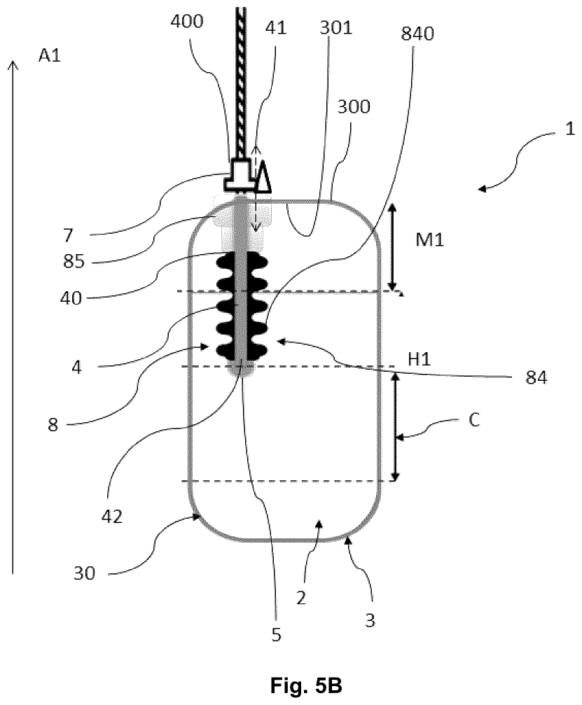

According to a first embodiment shown in FIGS. 5A and 5B, the hermetic isolating device 8 may be contained within the reaction container 3. Consequently, the hermetic isolating device 8 according to this first configuration is arranged so as to join the arm 4 to an inner face 301 of the wall 30 of the reaction container 3, the inner face 301 facing the reaction 2. Similarly, the seal 85 is arranged between the bellows 84 or the cylindrical pocket and the inner face 301 of the wall 30.

More specifically, as shown in FIG. 5A, the hermetic isolating device 8 is extendible along a travel C in the reaction container 3.

Furthermore, the arm 4 can move in translation along the first axis A1 to compress the hermetic isolating device 8, as shown in FIG. 5B. However, this translational movement is limited to a top position H1 in which the bellows 84 or the elastic pocket cannot be further compressed.

There is therefore a dead zone M1 representing a zone in which the sensor 5 cannot detect information and observe the parameter or parameters to be studied.

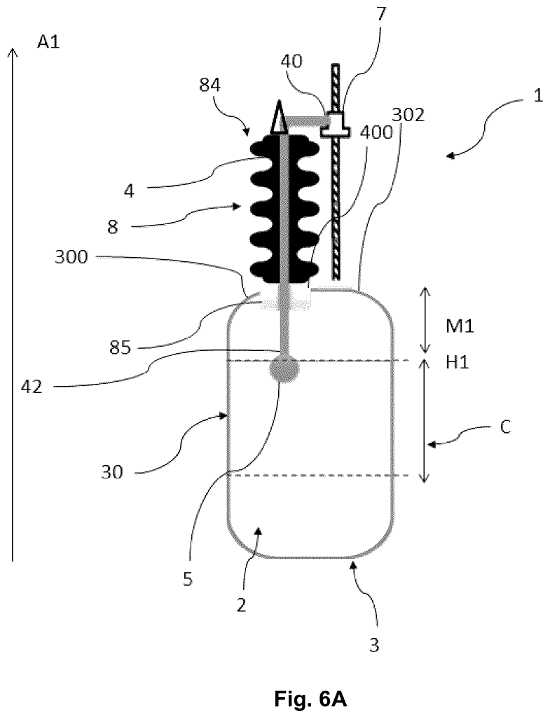

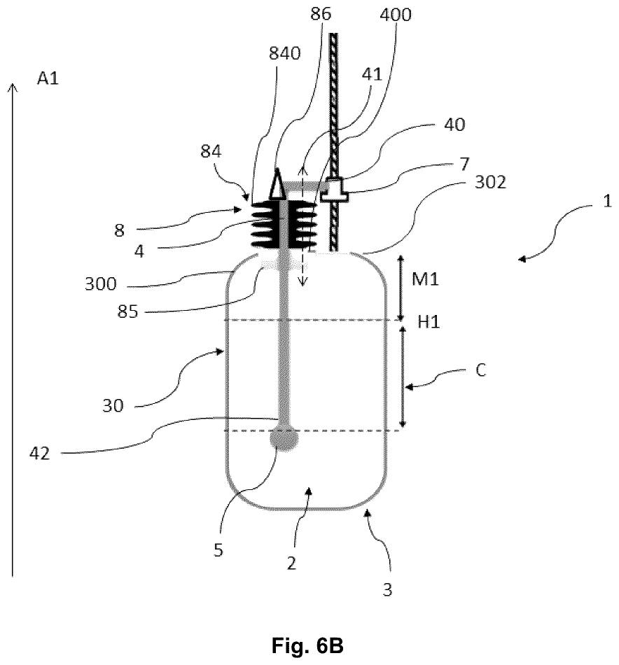

A second configuration of the analysis device 1 can therefore be envisaged, as shown in FIGS. 6A and 6B.

In this configuration, the hermetic isolating device 8 is arranged outside the reaction container 3. More specifically, the hermetic isolating device 8 is arranged so as to join the arm 4 to an outer face 302 of the wall 30 of the reaction container 3, the outer face 302 facing an environment outside the reaction 2 and being opposite the inner face 301. Similarly, the seal 85 is arranged between the bellows 84 or the cylindrical pocket and the outer face 302 of the wall 30.

This second configuration, with the hermetic isolating device 8 arranged outside the reaction container 3, has the advantage of increasing the travel C of the arm 4, thereby enabling the sensor 5 to observe the parameter or parameters over a larger height range, in the illustrated example.

To enable the bellows 84 or the elastic pocket to retain a natural shape during movement of the arm 4, the bellows 84 or the elastic pocket may be coupled to a vent 86 enabling any excess pressure inside the hermetic isolating device 8 to be discharged. The vent 86 may also be associated with a 0.2 μm filter to enable venting to atmospheric pressure without enabling contaminants to enter the hermetic isolating device 8.

Furthermore, the analysis device 1 according to the invention has the advantage of requiring just one fastening port, i.e. the opening 400 in the face 300 of the wall 30, for insertion of the arm 4 and of the sensor 5, and of being suited for assembly against the standard DN50 or DN70 clamp fastening port of the reaction containers 3 currently available on the market. Standard reaction containers 3 therefore do not need to be adapted to the analysis device 1.

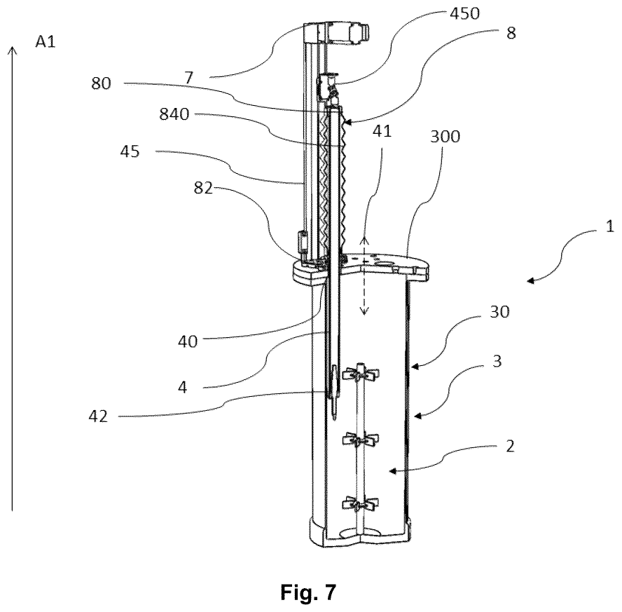

FIG. 7 is a detailed view of a device 1 for analyzing and monitoring at least one parameter of the chemical or biochemical or biological reaction 2 in the reaction container 3 according to the configuration shown in FIG. 6A and FIG. 6B. As specified above, the arm 4 has a degree of freedom in translation in relation to the wall 30 of the reaction container 3, such that the arm 4 moves in translation substantially perpendicularly in relation to the face 300 of the wall 30. This translational movement may for example be provided by a translational movement guide 45 joining the drive device 7 and the arm 4. Preferably, the translational movement guide 45 is a rail joined to the arm at a fastening 450. The fastening 450 is then moveable on the rail, i.e. the translational movement guide 45, and the arm 4 is fastened thereto, for example by push-fitting.

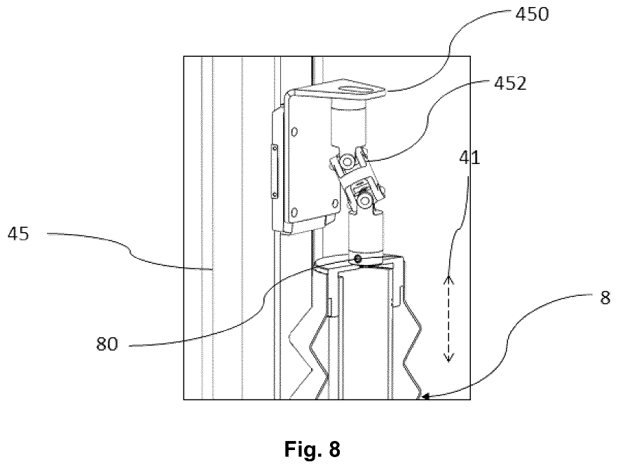

In a variant shown in FIG. 8, the fastening 450 may comprise a double universal joint 452, thus ensuring good alignment of the arm 4 and preventing overhang.

Furthermore, the sensor 5 may also be fastened to the free end 42 of the arm 4 by means of a flexible sealed connector, such as a PG13.5 connector.

1. A device for analyzing at least one parameter of a chemical or biochemical or biological reaction in a reaction container, the analysis device comprising:
an arm intended to be fastened by means of a sliding link against a face of a wall of the reaction container by means of a static end, the arm having a degree of freedom in translation in relation to the wall of the reaction container, a sensor designed to measure the at least one parameter, the sensor being fastened to a free end of the arm,
a drive device designed to cause an axial displacement of the arm along the degree of freedom,
a hermetic isolating device for isolating the arm from the chemical or biochemical or biological reaction, the hermetic isolating device joining the arm to the face of the wall of the reaction container.

2. The analysis device according to claim 1, wherein the hermetic isolating device is made of one of the following materials: silicone, a fluoroelastomer, polyether ether ketone (PEEK), ethylene propylene diene monomer (EPDM), polytetrafluoroethylene (PTFE).

3. The analysis device according to claim 1, wherein the hermetic isolating device comprises a bellows.

4. The analysis device according to claim 1, wherein the wall comprises an inner face facing the reaction, the hermetic isolating device being arranged to join the arm to the inner face of the wall.

5. The analysis device according to claim 1, wherein the wall comprises an outer face facing an environment outside the reaction, the hermetic isolating device being arranged to join the arm to the outer face of the wall.

6. The analysis device according to claim 1, comprising control means for the arm that are connected to the drive device and arranged to control the displacement of the arm along the degree of freedom.

7. The analysis device according to claim 1, comprising means for transmitting data that can be measured by the sensor.

8. The analysis device according to claim 1, comprising a device for recording the data measured by the sensor.

9. The analysis device according to claim 1, wherein the sensor is a sensor able to measure one of the following data: pH, dissolved oxygen (O₂), carbon dioxide (CO₂), electrical conductivity, redox potential or temperature.

10. The analysis device according to claim 1, wherein the arm is a telescopic arm.

11. The analysis device according to claim 3, wherein the hermetic isolating device comprises a seal positioned between the bellows and the face of the wall of the reaction container, the seal comprising a first sealing surface in contact with the face of the wall of the reaction container and a second sealing surface in contact with the bellows.

12. The analysis device according to claim 11, wherein the seal is a wiper seal.

13. The analysis device according to claim 1, wherein the hermetic isolating device comprises a guide element designed to prevent relative movement between the arm and the face of the wall of the reaction container in a plane parallel to said face.

14. A reaction container for a chemical or biochemical or biological reaction comprising the device for analyzing at least one parameter of the chemical or biochemical or biological reaction in the reaction container according to claim 1.
